(19) United States
(12) Patent Application Publication
(10) Pub. No.: US 2023/0172761 A1
(43) Pub. Date: Jun. 8, 2023

(54) SELF-ADHESIVE FLEXIBLE BANDAGES FOR JOINTS

(71) Applicant: Samuel Messinger, Ramot Bet Shemish (IL)

(72) Inventor: Samuel Messinger, Ramot Bet Shemish (IL)

(21) Appl. No.: 17/544,967

(22) Filed: Dec. 8, 2021

(51) Int. Cl. A61F 13/02; A61F 13/06; A61F 13/10

(52) U.S. Cl. CPC A61F 13/0273; A61F 13/061; A61F 13/101; A61F 13/105; A61F 2013/00119

(57) ABSTRACT

A self-adhesive flexible therapeutic bandage for joints is disclosed. The bandage has a permanent hole cut at a central portion for applying the bandage directly to a bodily injury and a plurality of adhesive fastening strips at each end of the bandage for protecting, supporting, and resisting the movement of the muscle-skeletal structure at the joints area. The strips are arranged in a crisscross manner for better protection. Each strip is capable of providing independent flexion.

### FIELD OF THE INVENTION

The present invention generally relates to adhesive bandages for joints at knees, elbows, and fingers which require an elastic bandage when injured to promote healing. More specifically, the present invention relates to adhesive bandages having a hole cut in a central portion of the bandage to promote movement and a plurality of adhesive fastening strips which is fastened in a crisscross manner to secure on rounded, contoured, and/or flexing areas of the body such as knees and elbows,

### BACKGROUND

A bandage is a piece of material used either to support a medical device such as a dressing or splint, or on its own to provide support to or to restrict the movement of a part of the body, When used with a dressing, the dressing is applied directly to an injury, and a bandage holds the dressing in place. Other bandages are used without dressings, such as elastic bandages that arc used to reduce swelling or provide support to a sprained ankle. Adhesive bandages typically include a pad of gauze or similar absorbent material positioned at a center of a strip of adhesive tape. When applied, the pad is positioned over an injury and secured to the skin via adhesive tape. Bandages, braces, casts, splints are a few of the many dressings used for knee and elbow joint injuries. Elastic bandages usually cover all the area and restrict blood flow which delays healing and recovery.

Few existing patent references are cited in the background as prior art over the presently disclosed subject matter and are explained as follows:

A prior art U.S. Pat. No. 10,463,544 B2 to Mark Joseph, entitled “Orthopedic system for immobilizing and supporting body parts” discloses a unitized cast system for immobilizing and supporting a body part. The casting systems use unitized multi-layer construction that uses dry-heat to soften the support layer to form a unitized structure to the body. The system can be removed by flexing open, re-heated, and re-molded as needed. This allows the cast to be easily reformed if it did not conform optimally, or if swelling increases or decreases. The cast can be simply removed, reheated, and then reformed on the patient to form a comfortable custom fit.

Another prior art KR 20140046881 A to Won Yong-kyung, entitled “A compression bandage for knee” discloses a compression bandage for knee where a user with a weak knee wearing the pressure bandage can easily stand up or climb stairs by receiving assist power from the pressure bandage when folds or stretches his or her knees. More specifically, the invention is to improve the wearing comfort by preventing the pressure bandage flows down or curling up the bottom of the pressure band even when wearing the pressure bandage for a long time. Though elastic bandages are commonly used to treat muscle sprains, they are uncomfortable and usually tight-fitting which may restrict movement and strains by reducing the flow of blood to a particular area by the application of even stable pressure which can restrict swelling at the place of injury.

There are disposable bandages with gauze-like pads and an adhesive tape strip that extends over the gauze pad so that the adhesive tape extensions can secure the bandage against the skin and cover the injury. For example, a prior art U.S. Pat. No. 7,605,299 B2 to Marc Zocher, entitled “Wound guard bandage” discloses a disposable bandage for covering a wound area of a skin surface. The bandage protects injuries, cuts, incisions, and abrasions along with providing limb movement and also serves as a shield for injury area. This bandage includes a first fastener portion and a second fastener portion, each having an underside surface provided with an adhesive, a dome structure having a generally rectilinear shape, and constructed from a rigid material that is constructed to provide flex.

Despite the presence of these bandages in the market, none of the above-mentioned prior art or otherwise provide a solution for an injury where the bandage not only provides compression around the injury but also does not restrict movement not resulting in reduced blood flow. The conventional bandages, due to their planar construction do not enable to from around an elbow or knee without wrinkling up and laterally falling off the place. However, these devices do not dispense adhesive bandages of the type under consideration here.

There is thus a widely recognized need for, and it would be highly advantageous to have, a lightweight bandage such as an adhesive bandage for knee and elbow joints configured with a hole cut in the central portion which serves to increase the blood circulation around the injury. Moreover, it is highly desirable to have an adhesive bandage that has multiple fastening edges which can be sealed in a crisscross overlapping pattern to help secure the bandage stay at its place without falling down. The bandage material like butyl neoprene and spacer fabric is used which can be expanded to treat the injured patient.

### SUMMARY OF THE INVENTION

The present invention generally discloses adhesive bandages for joints at knees, elbows, and fingers. Also, the present invention discloses adhesive bandages having a hole cut in a central portion of the bandage to promote movement and a plurality of adhesive fastening strips which is fastened in a crisscross manner to secure on rounded, contoured, and/or flexing areas of the body such as knees and elbows.

The present invention relates to self-adhesive flexible bandages or therapeutic bandages for various rounded and flexing areas of the human body such as joints at knees, joints at elbows, and joints at fingers, with a hole cut in the central portion of the tape and plurality of adhesive fastening edges/strips to secure the bandage on its place without bunching up. In on embodiment, the therapeutic bandage is configured to apply smoothly and evenly on flexing and/or rounded areas of the body such as joints at knees, joints at elbows, and joints at fingers. Furthermore, the plurality of adhesive fastening strips are secured on the body part in a crisscross manner as to not wrinkle up or peel off from its place. In one embodiment, the bandage is expandable to conform to the expansion and contraction of the point during movement.

In one embodiment, the present invention provides a single bandage for various parts of the body, thus eliminating the need to purchase different bandages for various injuries.

In one embodiment, the bandage is provided with a hollow cavity for promoting a movement of joints like knees, elbows, and fingers resulting in increased circulation.

In one embodiment, the bandage is designed with a die-cut in the central portion to provide compression and warmth around the injury without reducing the blood supply when it is trying to heal.

In one embodiment, the bandage is a prepackaged adhesive bandage with a release liner strip on down-facing side of the tape. Moreover, the bandage is formed of a fibrous or non-fibrous, woven or non-woven plastic material that is flexible, expandable, breathable, gas-permeable, and hydrophobic in nature.

In one embodiment, the shaped configuration of the adhesive bandage with multiple sealing edges forms a continuous lattice pattern, enabling it to be applied and fitted securely to a contoured body part. The fit is achieved because the self-adhesive tabs can be fastened and wrapped around the contoured edge at any degree to conform to any contour adjacent to the injury. In addition, this invention can provide relief from muscle strain, swelling, and joint pain like tendonitis knee and tendonitis elbow With offering turn support and compression along with warmth.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and structures disclosed herein. The description of a method step or a structure referenced by a numeral in a drawing is applicable to the description of that method step or structure shown by that same numeral in any subsequent drawing herein.

FIG. 1A shows a therapeutic bandage with a hole cut in the center in an embodiment of the present invention.

FIG. 1A1 shows a therapeutic bandage with a hole cut in the center with a plurality of fastening edges in another embodiment of present invention.

FIG. 1B shows a plurality of fastening edges of the therapeutic bandage with the hole cut in the center in one embodiment of the present invention.

FIG. 2A shows the application of the bandage on an injured area in a knee joint of the user in one embodiment of the present invention.

FIG. 2A1 shows the application of the bandage on the injured area in the knee joint of the user in another embodiment of the present invention.

FIG. 2B shows a plurality of fastening edges arranged in a crisscross manner over the backside of the knee joint of the user in one embodiment of the present invention.

FIG. 3 shows a side view of the therapeutic bandage describing the application of the therapeutic bandage over the user's knee joint in one embodiment of the present invention.

FIG. 4A shows a different type of therapeutic bandage with the hole cut in the center for the elbow joint in one embodiment of the present invention.

FIG. 4B shows pre-cut fastening, edges of the therapeutic bandage with the hole cut in the center in one embodiment of the present invention.

FIG. 5A shows the application of the therapeutic bandage with the adhesive fastening strips over the elbow of the user in another embodiment of the present invention.

FIG. 5B shows a side view of the therapeutic bandage describing the application of the therapeutic bandage over the user's elbow joint in one embodiment of the present invention.

FIG. 6 shows a therapeutic bandage with a hole cut in a center along with adhesive fastening strips in one embodiment of the present invention.

FIG. 7 shows the application of the therapeutic bandage with a plurality of adhesive fastening strips over the finger joint of the user in one embodiment of the present invention.

FIG. 8 shows the application of the therapeutic bandage with a plurality of fastening edges arranged in a crisscross manner over the backside of the finger joint of the user in one embodiment of the present invention.

FIG. 9 shows a side view of the therapeutic bandage describing the application of the therapeutic bandage over the user's finger joint in one embodiment of the present invention.

FIG. 10 shows multiple therapeutic bandages on a die-cut sheet for application over finger joint in one embodiment of the present invention.

### DETAILED DESCRIPTION OF EMBODIMENTS

The present invention is best understood by reference to the detailed figures and description set forth herein.

It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

The present invention broadly relates to a support for an area of a body. Broadly described, the support includes a breathable dual stretch power knit neoprene material therapeutic bandage compression with the area of the body and adhesive fastening strips for securing the bandage in its compression with the area of the body. The bandage has a permanent hole in its center to move the joints freely and adhesive fastening strips apply tension at points proximate to a perimeter of the bandage. In one embodiment, the bandage uses a breathable polyester and butyl neoprene material that increases warmth and compression to aid blood flow and healing of both the knee joint and elbow joints. Among other flexible materials, a waterproof and expandable spacer fabric may be used for bandages. Moreover, due to the adhesive fastening strips, the user may adjust the compression on the joints according to the needs. In one embodiment, the permanent hole of the present invention may be covered by gauze if the wound needs to be covered.

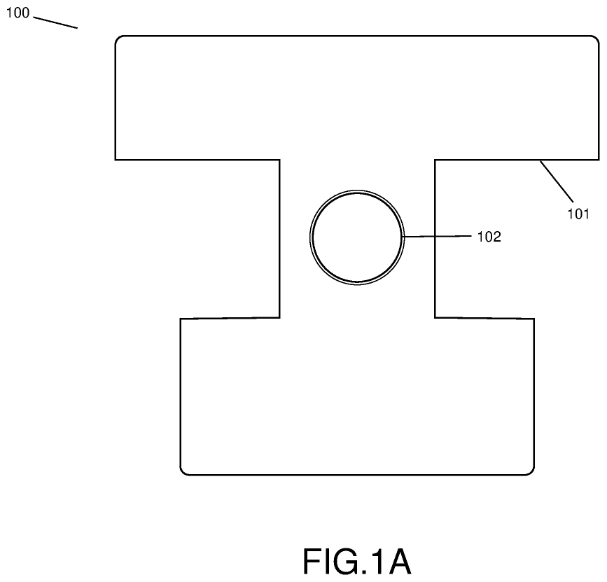

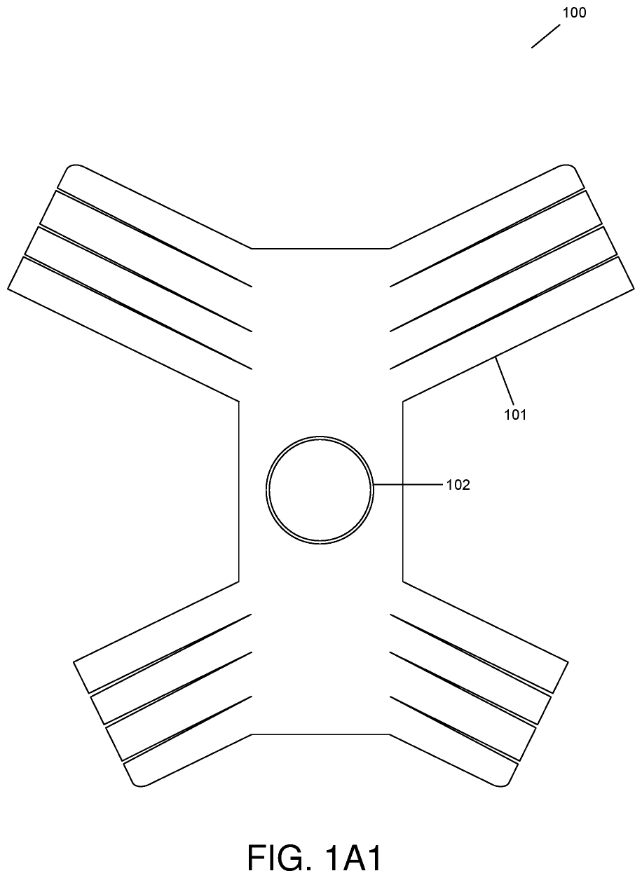

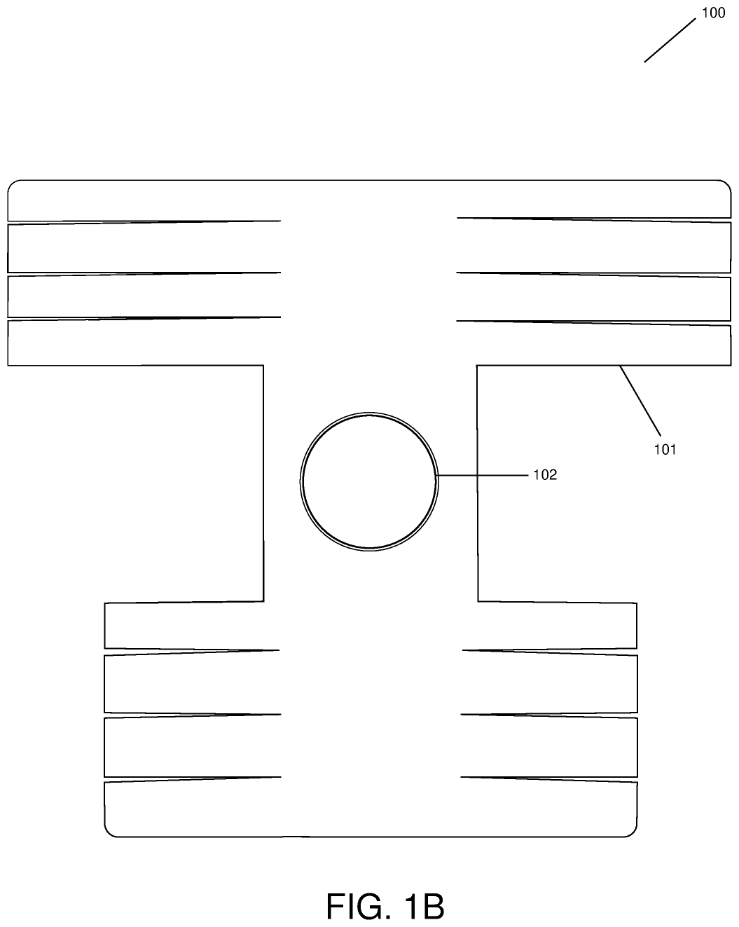

The present invention relates to self-adhesive flexible bandages or therapeutic bandages that can be used for knee, elbow, and finger joints configured with a hole cut in a central portion which serves to increase the blood circulation around the injury and is configured to flexion of the joint. Referring to FIGS. 1A to 3 which describes the therapeutic bandage or knee support 100, according to one embodiment of the present invention. In one embodiment, the therapeutic bandage 100 is of I-shape with extending wings at the ends of the bandage. The therapeutic bandage 100 has a multi-layer bandage body with an adhesive layer that is laminated on an inner surface of the multi-layer bandage body of the bandage 100. In one embodiment, the multi-layer main body of the bandage is made of breathable neoprene. In one embodiment, the multi-layer main body of the bandage is made of breathable neoprene.

Referring to FIG. 1A, the therapeutic bandage 100 with a permanent hole cut 102 in the center, according to one embodiment of the present invention. The hole cut 192 is situated in the center of the multi-layer bandage body of the bandage 100 so that the placement of the bandage 100 can be made easy on joints. In one embodiment, the hole cut 102 is located directly on the knee joint and facilitates flexion of the knee joint to minimize any chafing that might result from the bandage rubbing against the skin when the bandage 100 is worn for a long period of time. In one embodiment, the therapeutic bandage 100 is formed from a material including, but not limited to, elastic stretchy nylon, lycra, spandex, polyester, cloth, etc. The dimensions of the bandage 100 vary depending on its usage, for example, knee joint, elbow joint, etc.

Referring to FIG. 1A1, a therapeutic bandage 100 with a hole cut 102 in the center with a plurality of adhesive fastening edges/stripes/strips 101 at each end of the multi-layer bandage body, according to another embodiment of the present invention. The bandage 100 has a permanent hole 192 in its center to place the bandage 100 on the knee joint of the user. The plurality of adhesive fastening strips 101 are made on the extending wings of the bandage 100 and are made at an angle to a length of the bandage 100. In an embodiment, the plurality of fastening strips 101 are arranged in a manner to provide seamless fit over the knee joint of the user. In one embodiment, the plurality of adhesive fastening strips 101 are longer on the upper side of the bandage 100 due to the thickness of the upper leg while the lower portion of the adhesive fastening strips 101 are shorter.

Referring to FIG. 1B, the plurality of straight adhesive fastening strips 101 of the therapeutic bandage 100, according to one embodiment of the present invention. The straight adhesive fastening strips 101 are made on the extending wings of the bandage 100. The straight adhesive fastening strips 101 are made longitudinal to a length of the bandage 100. The bandage 100 has a permanent hole 102 in its center which can be placed on the joints of a user. The adhesive is laminated at the backside of the bandage 100, thereby placing the bandage 100 firmly on the injury. In one embodiment, the plurality of adhesive fastening strips 101 are used to tighten the bandage 100 on the joint.

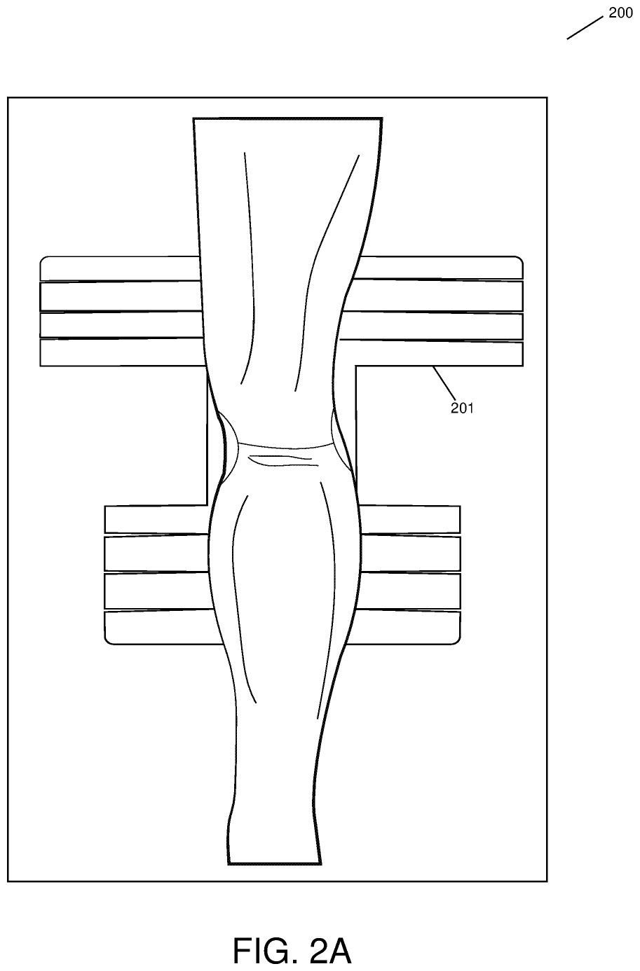

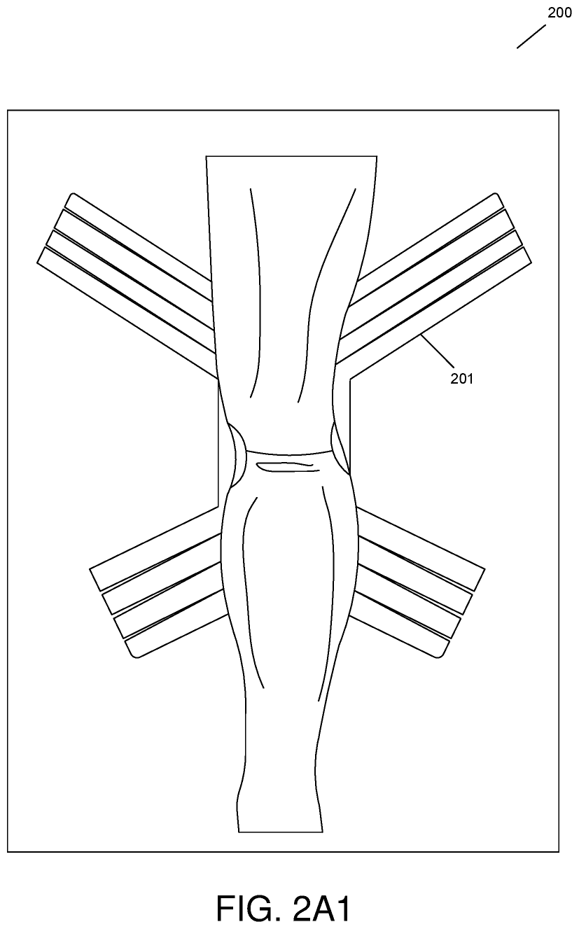

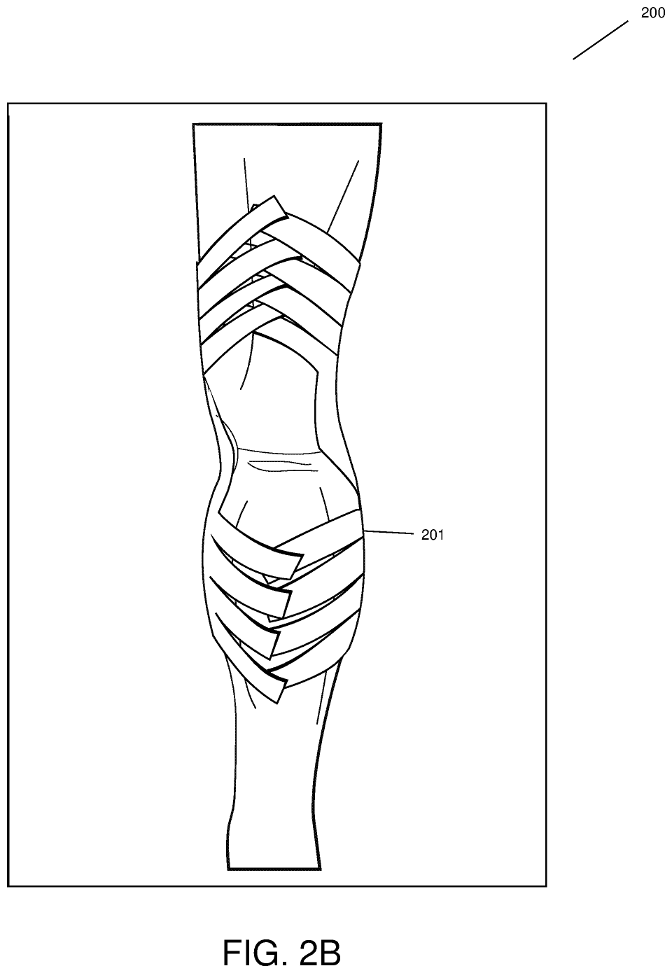

Referring to FIG. 2A, the application of the therapeutic bandage 200 on the user's knee joint, according to one embodiment of the present invention. The bandage 200 is placed on the knee joint of the user in a manner that the hole is situated on the joint of the user and fastening adhesive strips 201 are placed straight over and below the knee joint of the user. In one embodiment, the adhesive part which is the inner part of the bandage 100, is placed on the side of the knee joint.

Referring to FIG. 2A1, the application of the bandage 200 on the injured area in the knee joint of the user, according to another embodiment of the present invention. The bandage 200 has a plurality of adhesive fastening strips 201 that are arranged intersecting the knee of the user. The bandage 200 is placed in a manner wherein the hole in the center is placed over the knee joint of the user.

Referring to FIG. 2B, a crisscross arrangement of the plurality of adhesive fastening strips 201, according to one embodiment of the present invention. The fastening strips 201 are arranged crisscross on the backside of the knee joint of the user for strength and flexibility.

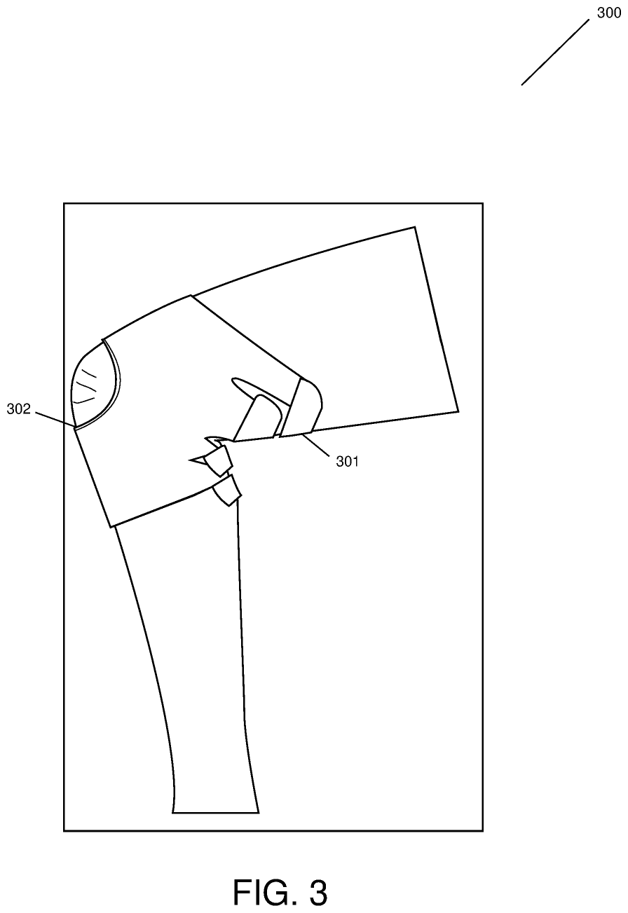

Referring to FIG. 3, a side view of the therapeutic bandage 300 that describes application of the therapeutic bandage 300 over the user's knee joint, according to one embodiment of the present invention. The therapeutic bandage 300 is applied in a manner wherein the hole 302 is just on the knee joint of the user and the plurality of fastening adhesive strips 301 are placed behind the knee joint on the calf muscles and thigh muscle of the user. In one embodiment, these adhesive fastening strips 301 are adjustable according to the size of the user's calf and thigh muscles.

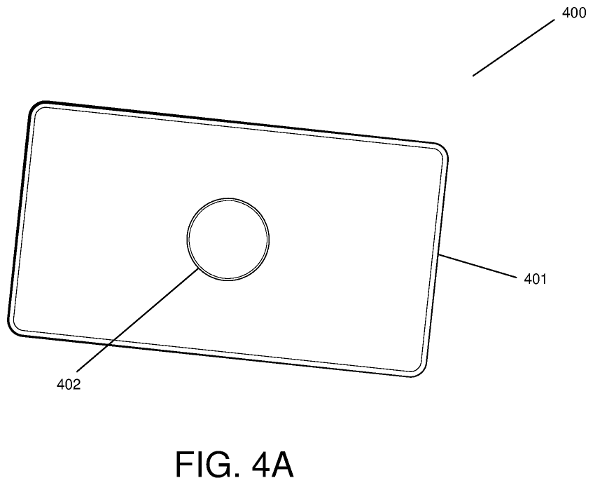

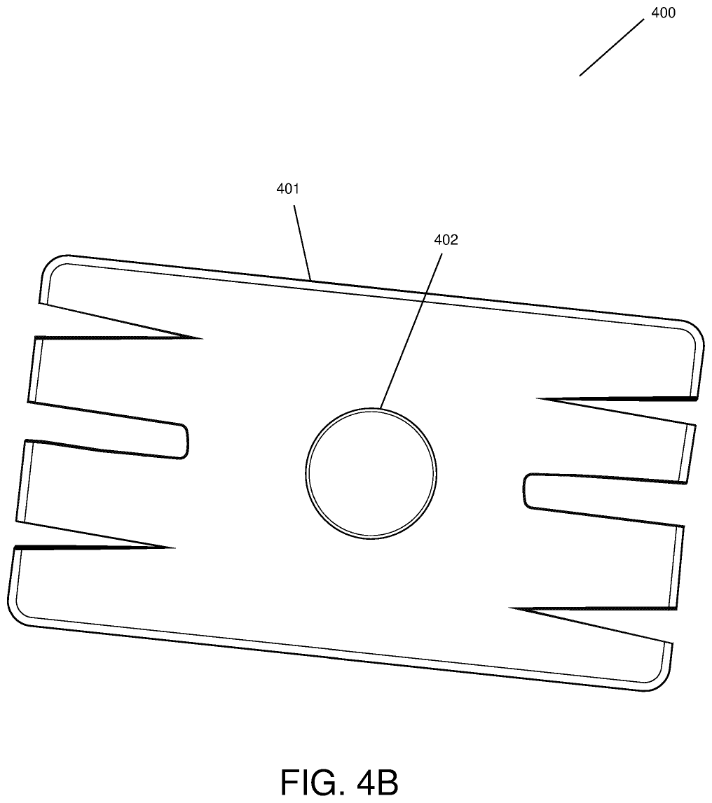

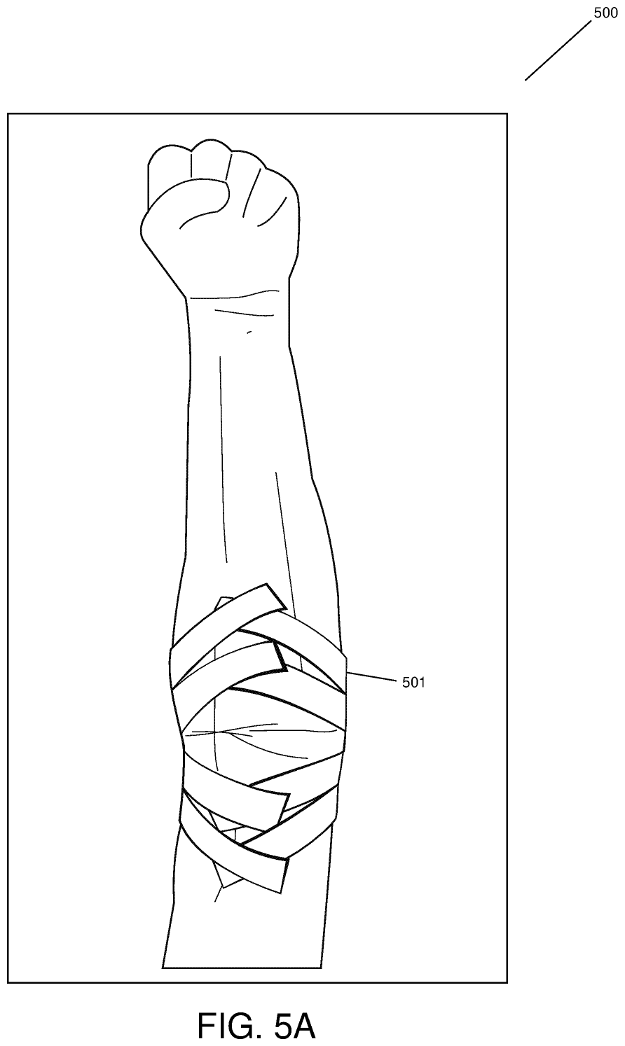

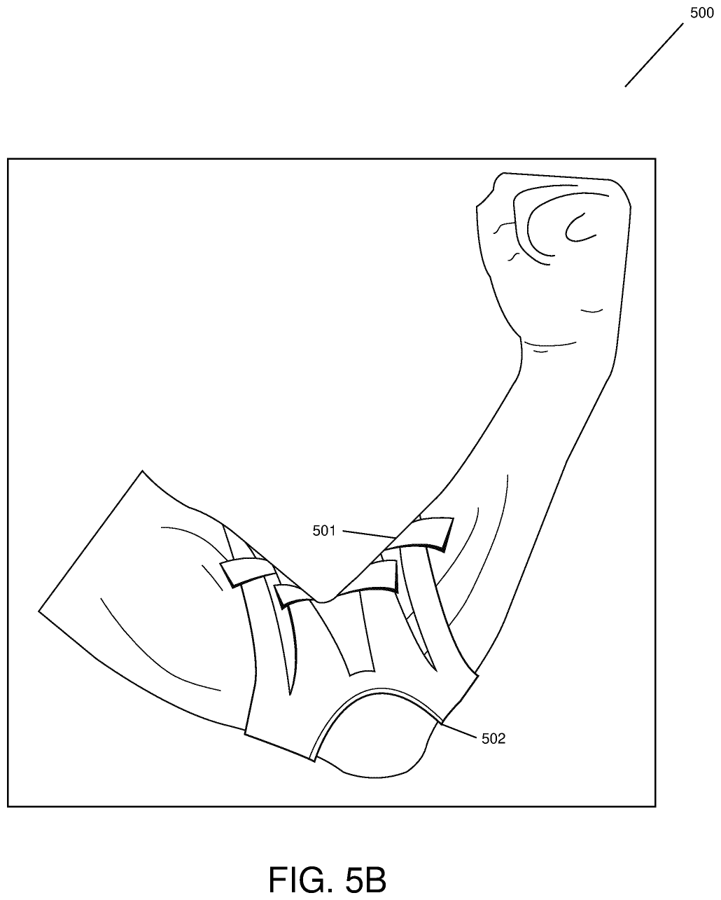

FIGS. 4A to 5B illustrate different types of therapeutic bandage 400 which can be applied to elbow joint of the user. The elbow bandage 400 has a hole in the center 402 so that the bandage can be placed on the joint for better movement. FIG. 4B illustrates the therapeutic bandage 400 with fastening strips 401. The hole cut 402 is situated in the center of the bandage 400 for better movement of the elbow joint. FIG. 5A illustrates the application of the elbow bandage 500 on the user's elbow joint. The fastening strips 501 are arranged in a manner that can hold the bandage 500 through a crisscross manner.

Referring to FIG. 5B a side view of the therapeutic bandage 500 describing the application of the therapeutic bandage 500 on the user's elbow joint, according to one embodiment of the present invention. The bandage 500 is applied to the elbow of the user in a manner that the hole 502 is situated on the elbow joint of the user and fastening strips 501 are stick to the above and lower part of the elbow joint of the user. In one embodiment, the therapeutic bandage 500 is used for tennis elbow, golfers' elbow, elbow tendinitis weightlifting, basketball, and other sports joint pain relief injury and for recovery and prevention of sports injuries.

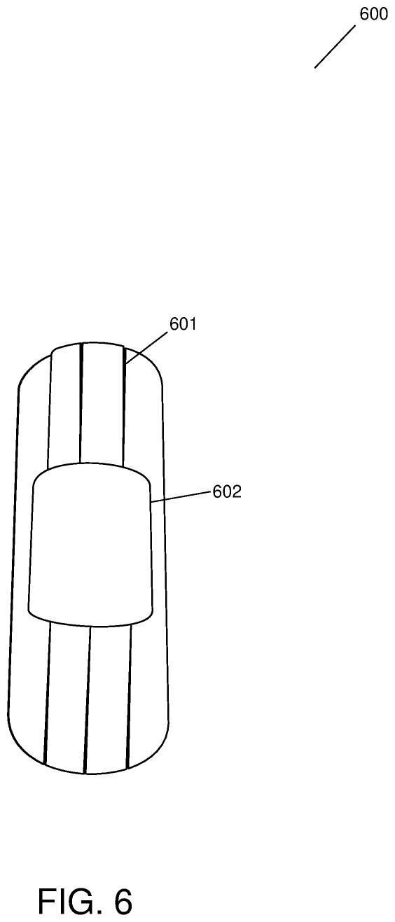

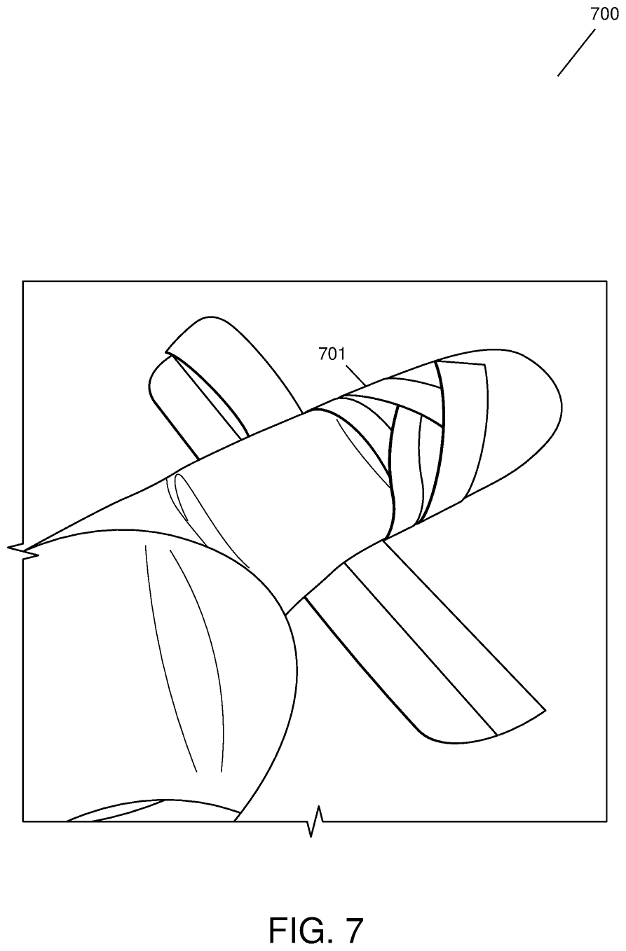

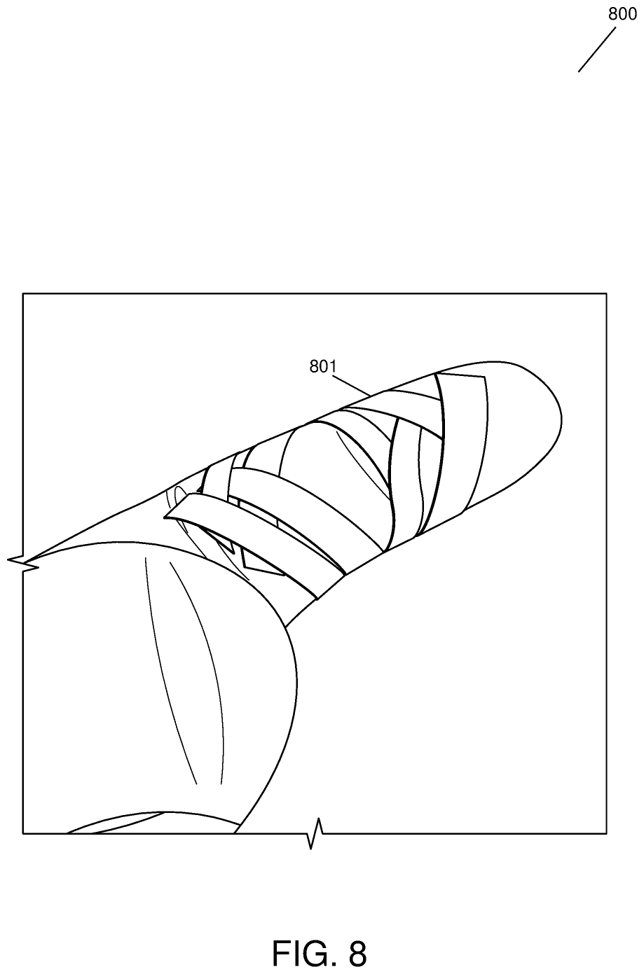

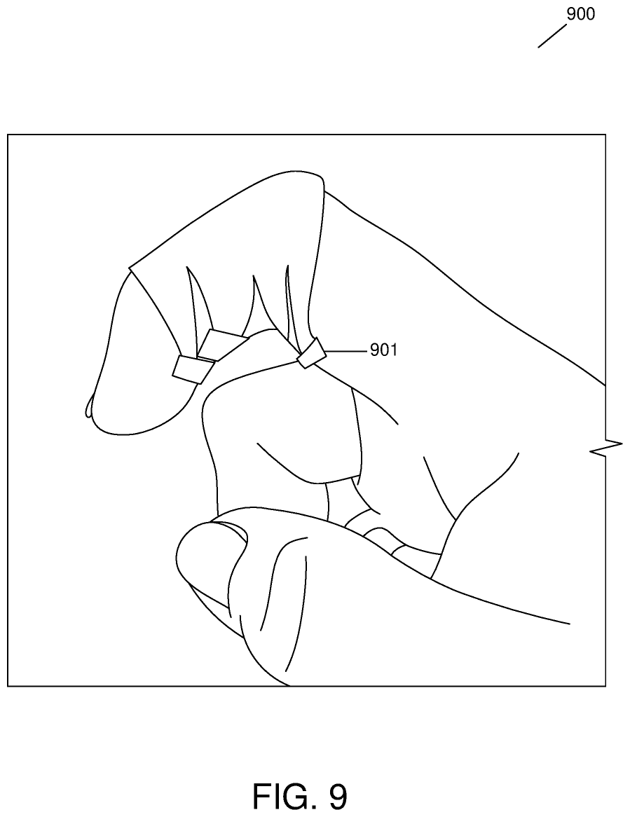

According to one embodiment, FIGS. 6-9 illustrate different types of therapeutic bandage which can be applied to the finger joint of the user. FIG. 6 illustrates the finger joint bandage 600 with a hole 602 in its center to place the bandage 600 over the injury of the user and pre-cut adhesive fastening strips 601 for flexibility of the fingers. According to another embodiment, the permanent hole of the finger joint bandage 602 may be covered by gauze if the wound needs to be covered. According to FIG. 7, which illustrates the application of the therapeutic bandage 700 in one embodiment of the present invention. The bandage 700 is placed on the front side of the finger and adhesive fastening strips 701 are wrapped around the backside of the finger in crisscross manner. According to FIG. 8, which illustrates the practical aspect of the therapeutic bandage 800. The adhesive fastening strips 801 of the therapeutic bandage 800 is applied in a manner forming a crisscross over the user's backside of the finger for better grip and protection. According to FIG. 9, which illustrates a side view of the user's finger with the therapeutic bandage 900. The overall bandage 900 and crisscross of the adhesive fastening strips 901 are arranged in a manner which provides maximum flexibility to the user while using the finger.

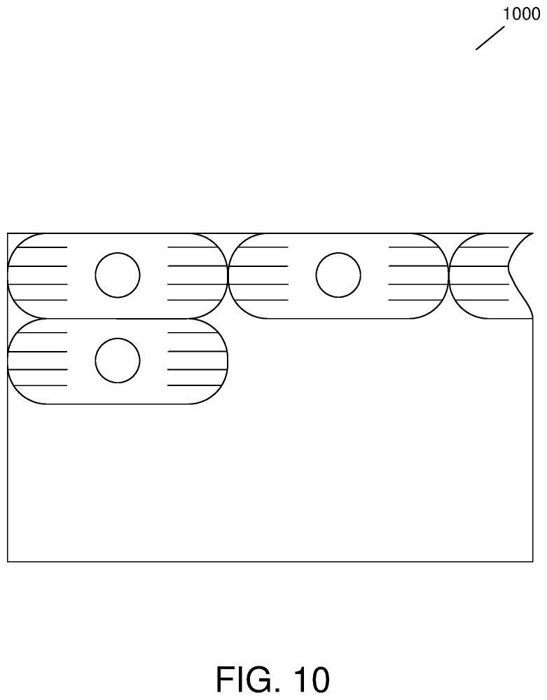

Referring to FIG. 10, multiple therapeutic bandages on a die-cut sheet 1000 for application over finger joint, according to one embodiment of the present invention. The manufacturing of the therapeutic bandages involves manufacturing multiple therapeutic bandages on the die-cut sheet 1000 and then removing them together before the use. In one embodiment, due to the flexibility of the bandage material and a ventilation hole in the center it draws perspiration away during movement and the ventilation hole is covered with anti-bacterial expandable material. In another embodiment, the bandage is washable and fits men's and women's knee or elbow joints. Moreover, the crisscross adhesive straps are adjustable to a specific body part and expandable to certain movements creating exact compression needed which is not achievable from other designs.

In one embodiment, the shaped configuration of the adhesive bandage with multiple sealing edges forms a continuous lattice pattern, enabling it to be applied and fitted securely to a contoured body part. The fit is achieved because the self-adhesive fastening strips are fastened and wrapped around the contoured edge at any degree to conform to any contour adjacent to the injury. In addition, the therapeutic bandage of the present invention provides relief from muscle strain, swelling and joint pain like tendonitis knee and tendonitis elbow with offering firm support and compression along with warmth.

Preferred embodiments of this invention are described herein, including the best mode known to the inventors for carrying out the invention. It should be understood that the illustrated embodiments are exemplary only and should not be taken as limiting the scope of the invention.

The foregoing description comprise illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings in the foregoing descriptions. Although specific terms may be employed herein, they are used only in generic and descriptive sense and not for purposes of limitation. Accordingly, the present invention is not limited to the specific embodiments illustrated herein.

What is claimed is:

1. A self-adhesive flexible bandage for joint, comprising:
a multi-layer bandage body with an adhesive layer laminated on an inner surface of the multi-layer bandage body;
a permanent hole cut at a center of the multi-layer bandage body and covered with an anti-bacterial expandable material; and
a plurality of adhesive fastening strips on an extending wing at each end of the multi-layer bandage body, wherein the plurality of adhesive fastening strips are made at an angle to a length of the bandage body, and wherein the plurality of adhesive fastening strips are adjustable.

2. The self-adhesive flexible bandage of claim 1, wherein the bandage is of I-shape configuration with the extending wings at the ends of the bandage.

3. The self-adhesive flexible bandage of claim 1, wherein the shape configuration of the bandage with the plurality of adhesive fastening strips forms a continuous lattice pattern to enable the bandage to be applied and fitted securely to a contoured body part.

4. The self-adhesive flexible bandage of claim 3, wherein the plurality of adhesive fastening strips are configured to be fastened and wrapped around a contoured edge at any degree to conform to any contour adjacent to an injury.

5. The self-adhesive flexible bandage of claim 1, wherein the bandage is for finger, knee, and elbow joints.

6. The self-adhesive flexible bandage of claim 1, wherein the permanent hole cut at the center is configured to flexion of the joint.

7. The self-adhesive flexible bandage of claim 1, wherein the plurality of adhesive fastening strips are longer on an upper side of the bandage and shorter on a lower side of the bandage.

8. The self-adhesive flexible bandage of claim 1, wherein the plurality of adhesive fastening strips tighten the bandage on the joints.

9. The self-adhesive flexible bandage of claim. 1, wherein the plurality of adhesive fastening strips are made on the extending wings of the bandage.

10. The self-adhesive flexible bandage of claim 1, wherein the plurality of adhesive fastening strips are made longitudinal to a length of the bandage.

11. The self-adhesive flexible bandage of claim 1, wherein the plurality of adhesive fastening strips are configured to wrap around the joint and fasten in a crisscross manner.

12. The self-adhesive flexible bandage of claim 1, wherein the multi-layer bandage body of the bandage is made of breathable neoprene.

13. The self-adhesive flexible bandage of claim 1, wherein the multi-layer bandage body of the bandage is made of a breathable polyester and butyl neoprene.

14. The self-adhesive flexible bandage of claim 1, wherein the bandage is made of elastic stretchy nylon, lycra, spandex, polyester, or cloth.

15. The self-adhesive flexible bandage of claim 1, wherein the permanent hole of the bandage is covered with gauze in case of wound injury.
